(19) United States
(12) Patent Application Publication
(10) Pub. No.: US 2015/0182436 A1
(43) Pub. Date: Jul. 2, 2015

(54) DEODORANT COMPOSITIONS HAVING ANTIBACTERIAL AND ODOR BLOCKING PROPERTIES AND METHODS FOR USING THE SAME

(71) Applicant: The Dial Corporation, Scottsdale, AZ (US)

(72) Inventor: Catherine Schmit, Glendale, AZ (US)

(21) Appl. No.: 14/144,666

(22) Filed: Dec. 31, 2013

(51) Int. Cl. A61K 8/43; A61K 8/27; A61K 8/26; A61K 8/42; A61K 8/33; A61K 8/34; A61K 8/35; A61K 8/41; A61K 8/28; A61Q 15/00

(52) U.S. Cl. CPC A61K 8/43; A61K 8/28; A61K 8/27; A61K 8/26; A61Q 15/00; A61K 8/33; A61K 8/347; A61K 8/35; A61K 8/34; A61K 8/416; A61K 8/42

(57) ABSTRACT

The present disclosure provides deodorant compositions having antibacterial and odor blocking properties and methods for using the deodorant compositions. In one embodiment, a deodorant product includes an odor blocking compound that reduces the perceived intensity of malodors or blocks malodors and an antibacterial compound effective to kill bacteria upon application thereto. In another embodiment, a method of deodorizing a malodor includes applying a deodorant product to the skin. The deodorant product includes an odor blocking compound that reduces the perceived intensity of malodors or blocks malodors and an antibacterial compound effective to kill bacteria upon application thereto.

### TECHNICAL FIELD

The present disclosure generally relates to personal care deodorant compositions, products, and processes for making and using the same. More particularly, the present disclosure relates to deodorant compositions having antibacterial and odor blocking properties and methods for using the deodorant compositions.

### BACKGROUND

Deodorant and antiperspirant compositions are well known personal care products used to prevent or eliminate perspiration and body odor caused by perspiration. The compositions come in a variety of forms and may be formulated, for example, into aerosols, pumps, sprays, liquids, roll-ons, lotions, creams, sticks, and soft solids, etc.

Deodorants are traditionally used to reduce or eliminate body odor caused by the breakdown and fermentation of secretions from apocrine sweat glands. Gram positive bacteria such as Corynebacterium xerosis, and Staphylococcus epidermis are primary examples of odor causing bacteria that inhabit human skin. Many commercially available deodorants help to eliminate body odor by acting as a bactericide or bacteriostat. Bactericides kill bacteria while bacteriostats inhibit the growth of bacteria.

There are various types of deodorant and antiperspirant compositions that are desirable by a large majority of the population because of their ease of application and the presence of compounds that prevent or block odors that accompany the secretion of perspiration. In one type, a composition is suspended in an anhydrous vehicle often including a solid water-insoluble wax. In a second type, a composition is dissolved in a liquid vehicle such as propylene glycol and gelled with a gelling agent such as dibenzylidene sorbitol. A third type includes an emulsion of an aqueous phase containing the composition and an oil phase containing, for example, a volatile silicone, fragrances, gellants, and other additives. In a fourth type for aerosol deodorant and antiperspirant products, a composition is suspended in an anhydrous liquid vehicle.

Personal care deodorants are typically formulated to work through a combination of fragrance technology and antibacterial activity. Most notably, a deodorant can be formulated to include a fragrance accord that helps mask the odor associated with human perspiration. Additionally, an antibacterial active may be incorporated in a deodorant composition to destroy or at least limit odor causing bacteria found on perspiring human skin. The most common antibacterial active for personal care deodorants has been 2,4,4′-trichloro-2′-hydroxy diphenyl ether (triclosan). Additionally, some glycols such as propylene glycol and dipropylene glycol function as bacteriostats. Triclosan and the glycols have found the most use in underarm deodorants because of their low cost, low irritancy and marked solubility in a host of carriers.

Current deodorant and antiperspirant products are primarily used after cleansing the axilla, for example after the user has taken a shower. However, there may be instances wherein odor elimination is desirable at other times, for example in between showers, before or during particularly stressful events, or before or during attempting physical activities, all of which situations are likely to cause increases in body odors. In this regard, there is a likelihood that any product applied under such circumstances would require the masking of body odor which has already formed prior to the application of the deodorant. Current deodorant and antiperspirant products may not be suitable for such uses, and if used therefor, may function only to “mask” or partially cover-up such body odors.

Accordingly, it is desirable to provide deodorant products that counteract the unwanted odors caused by bacteria in perspiration. Additionally, it is desirable to provide deodorant products that are suitable for application and use on skin that has not been recently cleaned, for example between showers. Still further, it is desirable to provide such deodorant products in an easy-to-apply form, such as aerosols, pumps, sprays, liquids, roll-ons, lotions, creams, sticks, and soft solids, etc. Furthermore, other desirable features and characteristics of the present disclosure will become apparent from the subsequent detailed description of the disclosure and the appended claims, taken in conjunction with the accompanying drawings and this background of the disclosure.

### BRIEF SUMMARY

The present disclosure provides deodorant compositions having antibacterial and odor blocking properties and methods for using the deodorant compositions. In one exemplary embodiment, a deodorant product includes an odor blocking compound that reduces the perceived intensity of malodors or blocks malodors and an antibacterial compound effective to kill bacteria upon application thereto.

In another exemplary embodiment, a method of deodorizing a malodor includes applying a deodorant product to the skin. The deodorant product includes an odor blocking compound that reduces the perceived intensity of malodors or blocks malodors and an antibacterial compound effective to kill bacteria upon application thereto.

This summary is provided to introduce a selection of concepts in a simplified form that are further described below in the detailed description. This summary is not intended to identify key features or essential features of the claimed subject matter, nor is it intended to be used as an aid in determining the scope of the claimed subject matter.

### DETAILED DESCRIPTION

The following detailed description of the disclosure is merely exemplary in nature and is not intended to limit the disclosure or the application and uses of the disclosure. Furthermore, there is no intention to be bound by any theory presented in the preceding background of the disclosure or the following detailed description of the disclosure.

The various embodiments contemplated herein relate to personal care deodorant products that exhibit antibacterial and odor blocking properties and methods for using the deodorant compositions. In an exemplary embodiment, the deodorant product includes at least an odor blocking composition and an antibacterial composition. Exemplary (non-limiting) odor blocking compositions and exemplary antibacterial compositions are provided in greater detail below in the paragraphs that follow. The odor blocking and antibacterial compositions may be provided in a deodorant product that further includes antiperspirant active compositions, fragrances, emollients, lubricants, and the like. The deodorant compositions may be provided in accordance with a plurality of formulations. For example, these formulations may include, but are not limited to, aerosols, pumps, sprays, liquids, roll-ons, lotions, creams, sticks, and soft solids, among others. Exemplary formulations for stick deodorants and aerosol deodorants are provided in greater detail below. Still further, methods for using the described deodorant compositions are provided in greater detail below.

### Odor Blocking Compositions

The term “odor blocking composition” as used herein refers to an item or product or cosmetic that may be used to reduce the perceived intensity or block malodor. Such odor blocking compositions may be distributed or dispersed in, on, or around the area of the locality by methods known to those in the art, such as by spraying or by evaporation of a liquid or solid containing the odor blocking composition. Suitable carriers are known to those skilled in the art and vary depending upon the method of distribution or dispersement of the deodorant. Exemplary odor blocking compositions are disclosed in U.S. Pat. No. 7,763,238, the contents of which are incorporated herein by reference in their entirety.

In general, the odor blocking compositions suitable for use in accordance with embodiments of the present disclosure block and/or reduce the perception of malodors, including body odors that originate from the axilla. Useful odor blocking compositions include agrumex (2-tert-butyl cyclohexyl acetate; 2-(1,1-dimethylethyl)-cyclohexanyl acetate); C14 aldehyde (1,4undecanolide; gamma-undecalactone); ambrettolide (cyclohexadec-6-olide; oxacycloheptadec-10-en-2-one; omega-6-hexadecenlactone; 6-hexadecanolide); anisyl aldehyde (4-methoxybenzaldehyde); CALONER1951 (7-methyl-3,4-dihydro-2H-1,5-benzodioxepin-3-one); 1-carvone (1-1menthyl-4-iso-propenyl-6-cyclohexen-2-one; carvol), CEDRAMBER® (cedryl methyl ether); citronellol 950 (3,7-dimethyl-6-octen-1-ol); citrylal (1,1-diethoxy-3,7-dimethyl-2,6-octadiene) as supplied by Haarmann & Reimer, Product No. 690980; CLARITONE® (2,4,4,7-tetramethyl-oct-6-en-3-one), cpd supra (15-pentadecalactone; cyclopentadecanolide supra; oxacyclohexadecan-2-one); α-damascone (1-(2,6,6-trimethyl-2-cyclohexen-1-yl)-2-buten-1-one); Δ-damascone (1-(2,6,6-trimethyl-3-cyclohexen-1-yl)-2-buten-1-one); datilat (Cyclohexyl methyl carbinol crotonate)dihydromyrcenol (3-methylenE-7-methyloctanol-7; 2,6-dimethyl-7-octen-2-ol); DYNASCONE® 10 (145,5-dimethyl-1-cyclohexen-1-yl)4-penten-1-one); ethyl vanillin (3-Ethoxy-4-hydroxybenzaldehyde); eugenol (2-methoxy-1-hydroxy-4-allylbenzene); evernyl(methyl 2,4-dihydroxy-3,6-dimethylbenzoate; 4-allyl-2-methoxyphenol; 4-allylguaiacol); FARENAL® (2,6,10-trimethyl-9-undecenal); floropal (2,4,6-trimethyl-4-phenyl-1,3-dioxane); GLOBALIDE® (oxacyclohexadecen-2-one); GLOBANONE® (cyclohexadecen-5-one-1; cyclohexadecenone); HEDIONE® (methyl dihydro jasmonate); cis-3-hexenol (cis-3-hexenol-1-ol); hexyl salicylate (hexyl-2-hydroxybenzoate); hexylcinnamic aldehyde (alpha-hexylcinnamaldehyde); α-ionone (4-(2,2,6-trimethyl-2cyclohexen-1-yl)-3-buten-2-one); β-ionone (4-(2,6,6-trimethyl-2cyclohexen-1-yl)-3-buten-2-one); ISO E SUPER® (octahydro-2,3,8,8-tetramethyl-2-acetonaphthone; and 2-acetyl-1,2,3,4,5,6,7,8-octahydro-2,3,8,8-tetramethyl naphthalene); isoananat (allyl cyclohexyloxyacetate); ISORALDEINE® 70 (alpha-n-methylionone; cyclocitrylidene methyl ethyl ketone); lilial (p-tert-butyl-alpha-methyldihydrocinnamaic aldehyde; 4-(1,1-dimethylethyl)-alpha-methylbenzenepropanal; p-t-bucinal), LYRAL® (4-(4-hydroxy-4-methylpentyl)-3-cyclohexenE-1-carboxaldehyde)); MAJANTOL® (2,2-dimethyl-3-(methylphenyl)propanol); menthyl acetate rf (4-menthyl-3-yl acetate); mugetanol (1-(4-isopropylcyclohexyl)ethanol); nerolione (1-(3-methyl-2-benzofuranyl)-ethanone); E-, Z-isomers (3:1) of 3-methyl-2-octenoic acid ethyl esters; H&R odor neutralizer (supplied by Haarmann and Reimer, Product No. D61012); oryclone special (4-tertbutylcyclohexyl acetate; 4-(1,1-dimethylethyl)cyclohexyl acetate); patchouli oil; phenylethyl alcohol; E-isomer of 3-methyl-2-pentenoic acid ethyl ester; E-isomer of 3-methyl-2-octenoic acid ethyl ester; Z-isomer of 3-methyl-2-pentenoic acid ethyl ester; rosaphen (2-methyl-5-phenylpentan-1-ol; tetrahydro-4-methyl-2-(2-methyl-1-propenyl)-2H-pyran); rose oxide hc (4-methyl-2-(2-methyl-1-propenyl)tetrahydro-2H-pyran); sandel (santalum album); sandolene (2-ethyl-4-(2,2,3-trimethyl-3-cyclopenten-1-yl)-2-buten-1-ol); tetrahydro linalool (3,7-Dimethyloctan-3-ol); timbranol (Isomethyltetrahydroionol); tonalide (7-Acetyl-1,1,3,4,4,6-hexamethyltetralin); and vertocitral (2,4-dimethyl-3-cyclohexene-1-carboxaldehyde).

In a preferred embodiment, odor blocking composition is provided that includes agrumex, C14 aldehyde, CEDRAMBER®, CLARITONE®, eugenol, Δ-damascone, dihydromyrcenol, dynascone 10, GLOBALIDE®, cis-3-hexenol, hexylcinnamic aldehyde, isoananat, menthyl acetate rf, oryclone special, phenylethyl alcohol, rosaphen, sandolene, tetrahydro linalool, or vertocitral, alone or in combination with other odor blocking composition(s). In another preferred embodiment, an odor blocking composition is provided that includes agrumex, C14 aldehyde, ambrettolide, anisyl aldehyde, CLARITONE®, dihydromyrcenol, dynascone 10, evernyl, GLOBALIDE®, hexyl salicylate, α-ionone, cpd supra, menthyl acetate rf, patchouli oil, sandolene, or vertocitral, alone or in combination with other odor blocking composition(s).

In a further embodiment of the present disclosure, methods of making an odor blocking composition include providing an odor blocking composition alone or in combination with other such agents in an amount effective to reduce perception of malodor is provided. Odor blocking compositions can be present in a deodorant composition from about 0.1% to about 10% by weight of the deodorant composition, in certain preferred embodiments.

### Antibacterial Compositions

An antibacterial agent is present in a composition of the present disclosure in an amount of about 0.1% to about 5%, and preferably about 0.2% to about 2%, by weight of the deodorant composition. To achieve the full advantage of the present disclosure, the antibacterial agent is present in an amount of about 0.3% to about 1%, by weight of the deodorant composition.

The antibacterial compositions can be ready to use compositions, which typically contain 0.1% to about 2%, preferably 0.15% to about 1.5%, and most preferably about 0.2% to about 1%, of an antibacterial agent, by weight of the composition. The antibacterial compositions also can be formulated as concentrates that are diluted before use with one to about 100 parts water or other diluent to provide an end use composition. The concentrated compositions typically contain greater than about 0.1% and up to about 5%, by weight, of the antibacterial agent. Applications also are envisioned wherein the end use composition contains greater than 2%, by weight, of the antibacterial agent.

As discussed above, the absolute amount of antibacterial agent present in the composition is not as important as the amount of available antibacterial agent in the composition. The amount of available antibacterial agent in the composition is related to the identity of the antibacterial agent in the composition, the amount of antibacterial agent in the composition, and the presence of optional ingredients in the composition.

The desired bacteria kill is achieved in a short contact time, for example 15 to 60 seconds. The composition also provides a persistent antibacterial efficacy.

Antibacterial agents useful in the present disclosure are exemplified by the following classes of compounds used alone or in combination:

### (1) Phenolic Antibacterial Agents

(a) 2-Hydroxydiphenyl Compounds

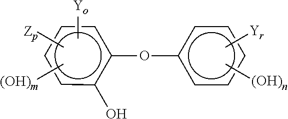

wherein Y is chlorine or bromine, Z is SO₃H, NO₂, or C₁-C₄ alkyl, r is 0 to 3, o is 0 to 3, p is 0 or 1, m is 0 or 1, and n is 0 or 1.

In preferred embodiments, Y is chlorine or bromine, m is 0, n is 0 or 1, o is 1 or 2, r is 1 or 2, and p is 0. In especially preferred embodiments, Y is chlorine, m is 0, n is 0, o is 1, r is 2, and p is 0.

A particularly useful 2-hydroxydiphenyl compound has a structure:

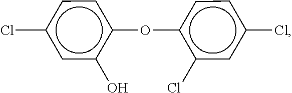

having the adopted name, triclosan, and available commercially under the tradename IRGASAN DP300, from BASF SE of Ludwigshafen, Germany. Another useful 2-hydroxydiphenyl compound is 2,2′-dihydroxy-5,5′-di-bromo-diphenyl ether.

(b) Phenol Derivatives

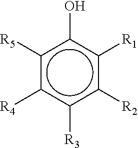

wherein R₁ is hydro, hydroxy, C₁-C₄ alkyl, chloro, nitro, phenyl, or benzyl; R₂ is hydro, hydroxy, C₁-C₆ alkyl, or halo; R₃ is hydro, C₁-C₆ alkyl, hydroxy, chloro, nitro, or a sulfur in the form of an alkali metal salt or ammonium salt; R₄ is hydro or methyl; and R₅ is hydro or nitro; Halo is bromo or, preferably, chloro.

Specific examples of phenol derivatives include, but are not limited to, chlorophenols (o-, m-, p-), 2,4-dichlorophenol, p-nitrophenol, picric acid, xylenol, p-chloro-m-xylenol, cresols (o-, m-, p-), p-chloro-m-cresol, pyrocatechol, resorcinol, 4-n-hexyl-resorcinol, pyrogallol, phloroglucin, carvacrol, thymol, p-chlorothymol, o-phenylphenol, o-benzylphenol, p-chloro-o-benzylphenol, phenol, 4-ethylphenol, and 4-phenolsul-fonic acid. Other phenol derivatives are listed in U.S. Pat. No. 6,436,885, incorporated herein by reference.

(c) Diphenyl Compounds

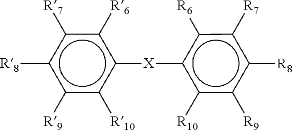

wherein X is sulfur or a methylene group, R₆ and R′₆ are hydroxy, and R₇, R′₇, R₈, R′₈, R₉, R′₉, R₁₀, and R′₁₀, independent of one another, are hydro or halo. Specific, non-limiting examples of diphenyl compounds are hexachlorophene, tetrachlorophene, dichlorophen, 2,3-dihydroxy-5,5′-dichlorodiphenyl sulfide, 2,2′-dihydroxy-3,3′,5,5′-tetrachlorodiphenyl sulfide, 2,2′-dihydroxy-3,5′,5,5′,6,6′-hexachlorodiphenyl sulfide, and 3,3′-di-bromo-5,5′-dichloro-2,2′-dihydroxydiphenylamine. Other diphenyl compounds are listed in U.S. Pat. No. 6,436,885, incorporated herein by reference.

### (2) Quaternary Ammonium Antibacterial Agents

Useful quaternary ammonium antibacterial agents have a general structural formula:

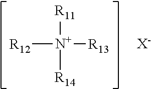

wherein at least one of R₁₁, R₁₂, R₁₃, and R₁₄ is an alkyl, aryl, or alkaryl substituent containing 6 to 26 carbon atoms. Alternatively, any two of the R substituents can be taken together, with the nitrogen atom, to form a five- or six-membered aliphatic or aromatic ring. Preferably, the entire ammonium cation portion of the antibacterial agent has a molecular weight of at least 165.

The substituents R₁₁, R₁₂, R₁₃, and R₁₄ can be straight chained or can be branched, but preferably are straight chained, and can include one or more amide, ether, or ester linkage. In particular, at least one substituent is C₆-C₂₆alkyl, C₆-C₂₆alkoxyaryl, C₆-C₂₆alkaryl, halogen-substituted C₆-C₂₆alkaryl, C₆-C₂₆alkylphenoxyalkyl, and the like. The remaining substituents on the quaternary nitrogen atom other than the above-mentioned substituent typically contain no more than 12 carbon atoms. In addition, the nitrogen atom of the quaternary ammonium antibacterial agent can be present in a ring system, either aliphatic, e.g., piperidinyl, or aromatic, e.g., pyridinyl. The anion X can be any salt-forming anion which renders the quaternary ammonium compound water soluble. Anions include, but are not limited to, a halide, for example, chloride, bromide, or iodide, methosulfate, and ethosulfate.

Preferred quaternary ammonium antibacterial agents have a structural formula:

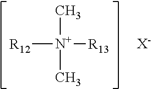

wherein R₁₂ and R₁₃, independently, are C₈-C₁₂alkyl, or R₁₂ is C₁₂-C₁₆alkyl, C₈-C₁₈alkylethoxy, or C₈-C₁₈alkylphenylethoxy, and R₁₃ is benzyl, and X is halo, methosulfate, ethosulfate, or p-toluenesulfonate. The alkyl groups R₁₂ and R₁₃ can be straight chained or branched, and preferably are linear.

The quaternary ammonium antibacterial agent in a present composition can be a single quaternary ammonium compound, or a mixture of two or more quaternary ammonium compounds. Particularly useful quaternary ammonium anti-microbial agents include dialkyl(C₈-C₁₀) dimethyl ammonium chlorides (e.g., dioctyl dimethyl ammonium chloride), alkyl dimethyl benzyl ammonium chlorides (e.g., benzalkonium chloride and myristyl dimethylbenzyl ammonium chloride), alkyl methyl dodecyl benzyl ammonium chloride, methyl dodecyl xylene-bis-trimethyl ammonium chloride, benzethonium chloride, dialkyl methyl benzyl ammonium chloride, alkyl dimethyl ethyl ammonium bromide, and an alkyl tertiary amine. Polymeric quaternary ammonium compounds based on these monomeric structures also can be used in the present disclosure. One example of a polymeric quaternary ammonium compound is POLYQUAT®, e.g., a 2-butenyl dimethyl ammonium chloride polymer. The above quaternary ammonium compounds are available commercially under the tradenames BARDAC®, BTC®, HYAMINE®, BARQUAT®, and LONZABAC®, from suppliers such as Lonza, Inc., Fairlawn, N.J. and Stepan Co., Northfield, Ill.

Additional examples of quaternary ammonium antibacterial agents include, but are not limited to, alkyl ammonium halides, such as cetyl trimethyl ammonium bromide; alkyl aryl ammonium halides, such as octadecyl dimethyl benzyl ammonium bromide; N-alkyl pyridinium halides, such as N-cetyl pyridinium bromide; and the like. Other suitable quaternary ammonium antibacterial agents have amide, ether, or ester moieties, such as octylphenoxyethoxy ethyl dimethyl benzyl ammonium chloride, N-(laurylcocoaminoformylmethyl)pyridinium chloride, and the like. Other classes of quaternary ammonium anti-microbial agents include those containing a substituted aromatic nucleus, for example, lauryloxyphenyl trimethyl ammonium chloride, cetylaminophenyl trimethyl ammonium methosulfate, dodecylphenyl trimethyl ammonium methosulfate, dodecylbenzyl trimethyl ammonium chloride, chlorinated dodecylbenzyl trimethyl ammonium chloride, and the like.

Specific quaternary ammonium antibacterial agents include, but are not limited to, behenalkonium chloride, cetalkonium chloride, cetarylalkonium bromide, cetrimonium tosylate, cetyl pyridinium chloride, lauralkonium bromide, lauralkonium chloride, lapyrium chloride, lauryl pyridinium chloride, myristalkonium chloride, olealkonium chloride, and isostearyl ethyldimonium chloride. Preferred quaternary ammonium antibacterial agents include benzalkonium chloride, benzethonium chloride, cetyl pyridinium bromide, and methylbenzethonium chloride.

### (3) Anilide and Bisguanidine Antibacterial Agents

Useful anilide and bisguanadine antibacterial agents include, but are not limited to, triclocarban, carbanilide, salicylanilide, tribromosalan, tetrachlorosalicylanilide, fluorosalan, chlorhexidine gluconate, chlorhexidine hydrochloride, and mixtures thereof.

### Deodorant Formulations

As noted above, deodorants including the above-noted odor-eliminating compositions and antibacterial compositions may be formulated broadly is any of easy-to-apply form, such as aerosols, pumps, sprays, liquids, roll-ons, lotions, creams, sticks, and soft solids, etc. Exemplary formulations for sticks and aerosols are provided herein for purposes of illustration, but are not intended to be limiting on the presently described embodiments.

In one embodiment, a deodorant composition of the present disclosure may be formulated as a stick deodorant. The stick deodorant formulation may include an anhydrous, hydrophobic vehicle, which includes a volatile silicone and/or high melting point component, and the above-noted odor blocking and antibacterial compositions suspended in the anhydrous, hydrophobic vehicle. The odor blocking and antibacterial compositions are preferably in the effective amounts described above.

The high melting point components may include any material suitable for use in an deodorant stick that melts at a temperature of about 70° C. or higher. Typical of such materials are the high melting point waxes. These include beeswax, spermaceti, carnauba, bayberry, candelilla, montan, ozokerite, ceresin, paraffin waxes, semi-microcrystalline and microcrystalline waxes, hydrogenated jojoba oil, and hydrogenated castor oil (castor wax). The preferred wax is hydrogenated castor oil. Other suitable high melting point components include various types of high melting point gelling agents such as polyethylene-vinyl acetate copolymers, polyethylene homopolymers, 12-hydroxystearic acid, and substituted and unsubstituted dibenzylidene alditols. Typically, the high melting point components include about 1 to about 25 wt. %, preferably about 2 to about 15 wt. %, of the composition. Volatile silicones include cyclomethicones and dimethicones, discussed above.

Other components may include, for example, non-volatile silicones, polyhydric alcohols having 3-6 carbon atoms and 2-6 hydroxy groups, fatty alcohols having from 12 to 24 carbon atoms, fatty alcohol esters, fatty acid esters, fatty amides, non-volatile paraffinic hydrocarbons, polyethylene glycols, polypropylene glycols, polyethylene and/or polypropylene glycol ethers of C₄-C₂₀ alcohols, polyethylene and/or polypropylene glycol esters of fatty acids, and mixtures thereof. The term “fatty” is intended to include hydrocarbon chains of about 8 to 30 carbon atoms, preferably about 12 to 18 carbon atoms.

Non-volatile silicones include polyalkylsiloxanes, polyalkylaryl siloxanes, and polyethersiloxanes with viscosities of about 5 to about 100,000 centistokes at 25° C., polymethylphenylsiloxanes with viscosities of about 15 to about 65 centistokes, and polyoxyalkylene ether dimethylsiloxane copolymers with viscosities of about 1200 to about 1500 centistokes.

Useful polyhydric alcohols include propylene glycol, butylenes glycol, dipropylene glycol and hexylene glycol. Fatty alcohols include stearyl alcohol, cetyl alcohol, myristyl alcohol, oleyl alcohol, and lauryl alcohol. Fatty alcohol esters include myristyl lactate, C₁₂₋₁₅ alcohols benzoate, cetyl acetate, and myristyl octanoate. Fatty acid esters include isopropyl palmitate, myristyl myristate, and glyceryl monostearate. Fatty amides include stearamide MEA, stearamide MEA-stearate, lauramide DEA, and myristamide MIPA.

Non-volatile paraffinic hydrocarbons include mineral oils and branched chain hydrocarbons with about 16 to 68, preferably about 20 to 40, carbon atoms. Suitable polyethylene glycols and polypropylene glycols will typically have molecular weights of about 500 to 6000, such as PEG-10, PEG-40, PEG-150 and PPG-20, often added as rheology modifiers to alter product appearance or sensory attributes.

Polyethylene and/or polypropylene glycol ethers or C₄-C₂₀ alcohols include PPG-10 butanediol, PPG-14 butyl ether, PPG-5-buteth-7, PPG-3-isostearth-9, PPG-3-myreth-3, oleth-10, and steareth-20. Polyethylene and/or polypropylene glycol esters of fatty acids include PEG-8 distearate, PEG-10 dioleate, and PPG-26 oleate. These are generally added to give emollient properties.

The deodorant product contemplated herein also may include additives, such as those used in conventional deodorants. For example, in addition to, or instead of, deodorant efficacy, the deodorant product may include additives that cause the deodorant product to exhibit long-lasting fragrance, odor protection, and/or another desired purpose and/or function. These additives include, but are not limited to, fragrances, including encapsulated fragrances, dyes, pigments, preservatives, antioxidants, moisturizers, and the like. These optional ingredients can be included in the deodorant product in an amount of about 0 to about 20 wt. %.

The above list of materials is by way of example only and is not intended to be a comprehensive list of all potential components of the antiperspirant products contemplated herein. Other high and low melting waxes, volatile and non-volatile compounds and other suitable components are readily identifiable to those skilled in the art. Of course, other ingredients such as colloidal silica, fumed silica, particulate polyolefins, talcum materials, colorants and preservatives may also be included as desired. For example, the composition may include up to about 10% fragrance or about 2% colorant by weight.

As noted initially above, in addition to the odor blocking and the antibacterial compositions, the deodorant formulation may include an active antiperspirant compound. The active antiperspirant compounds contain at least one active ingredient, typically metal salts, that are thought to reduce perspiration by diffusing through the sweat ducts of apocrine glands (sweat glands responsible for body odor) and hydrolyzing in the sweat ducts, where they combine with proteins to form an amorphous metal hydroxide agglomerate, plugging the sweat ducts so perspiration cannot diffuse to the skin surface. Some active antiperspirant compounds that may be used include astringent metallic salts, especially inorganic and organic salts of aluminum, zirconium, and zinc, as well as mixtures thereof. Particularly preferred are aluminum-containing and/or zirconium-containing salts or materials, such as aluminum halides, aluminum chlorohydrates, aluminum hydroxyhalides, zirconyl oxyhalides, zirconyl hydroxyhalides, and mixtures thereof. Exemplary aluminum salts include those having the general formula Al₂(OH)ₐCl_{b}×(H₂O), wherein a is from 2 to about 5; the sum of a and b is about 6; x is from about 1 to about 6; and wherein a, b, and x may have non-integer values. Exemplary zirconium salts include those having the general formula ZrO(OH)₂₋ₐClₐ×(H₂O), wherein a is from about 1.5 to about 1.87, x is from about 1 to about 7, and wherein a and x may both have non-integer values. Particularly preferred zirconium salts are those complexes that additionally contain aluminum and glycine, commonly known as ZAG complexes. These ZAG complexes contain aluminum chlorohydroxide and zironyl hyroxy chloride conforming to the above-described formulas. Examples of active antiperspirant compounds suitable for use in the various embodiments contemplated herein include aluminum dichlorohydrate, aluminum-zirconium octachlorohydrate, aluminum sesquichlorohydrate, aluminum chlorohydrex propylene glycol complex, aluminum dichlorohydrex propylene glycol complex, aluminum sesquichlorohydrex propylene glycol complex, aluminum chlorohydrex polyethylene glycol complex, aluminum dichlorohydrex polyethylene glycol complex, aluminum sesquichlorohydrex polyethylene glycol complex, aluminum-zirconium trichlorohydrate, aluminum zirconium tetrachlorohydrate, aluminum zirconium pentachlorohydrate, aluminum zirconium octachlorohydrate, aluminum zirconium trichlorohydrex glycine complex, aluminum zirconium tetrachlorohydrex glycine complex, aluminum zirconium pentachlorohydrex glycine complex, aluminum zirconium octachlorohydrex glycine complex, zirconium chlorohydrate, aluminum chloride, aluminum sulfate buffered, and the like, and mixtures thereof. In a preferred embodiment, the antiperspirant compound is aluminum zirconium trichlorohydrex. In another embodiment, the antiperspirant product includes an active antiperspirant compound present in the amount of 0 to about 25 wt. % (USP). As used herein, weight percent (USP) or wt. % (USP) of an antiperspirant salt is calculated as anhydrous weight percent in accordance with the U.S.P. method, as is known in the art. This calculation excludes any bound water and glycerin.

In another exemplary formulation, as noted above, the deodorant product may be formulated as an aerosol. In this embodiment, the deodorant composition has an anhydrous aerosol formulation, which is preferably substantially free of water and is stored in a container. The aerosol formulation includes the odor blocking compositions, the antibacterial compositions, and includes other various ingredients as discussed in the foregoing paragraphs, such as, for example, an active antiperspirant compound, talc, a volatile silicone, and one or more fragrances, and further includes a gaseous propellant, such as, one or more types of light hydrocarbons and/or fluorocarbons. The deodorant product is contained in the container under pressure from the gaseous propellant. With regard to the container under pressure, when a plunger cap is depressed, a valve stem is actuated against a spring to an open position allowing the liquid deodorant product to be forced, via a gaseous propellant, through a tube, the valve stem and finally out of a small hole in the plunger cap as an aerosol or mist. Other suitable aerosol valve-container arrangements known to those skilled in the art may also be used with the various embodiments of the present disclosure.

While two exemplary deodorant formulation embodiments have been provided in detail, it will be appreciated that the deodorant product may be formulated, using the above-described odor blocking and antimicrobial compositions (and other option ingredients as described above, such as active antiperspirants), according to any one of aerosols, pumps, sprays, liquids, roll-ons, lotions, creams, sticks, and soft solids, among others. The skilled artisan will readily be able to provide such formulations based on the present disclosure and the level of ordinary skill in the art.

### Methods of Use

As initially noted above, embodiments of the present disclosure are suitable for use applied to either cleaned or uncleaned skin, particularly in the axilla region. However, it is envisioned that the described embodiments will find particular application for use on uncleaned skin, for example between showers.

For example, current deodorant and antiperspirant products are primarily used after cleansing the axilla, for example after the user has taken a shower. However, there may be instances wherein odor elimination is desirable at other times, for example in between showers, before or during particularly stressful events, or before or during attempting physical activities, all of which situations are likely to cause increases in body odors. In this regard, there is a likelihood that any product applied under such circumstances would require the masking of body odor which has already formed prior to the application of the deodorant. The presently described deodorant formulations provide deodorant products that are suitable for application and use on skin that has not been recently cleaned, for example between showers. The deodorant products counteract the unwanted odors caused by bacteria in perspiration.

For example, it is anticipated that a user of the described deodorant products may anticipate a particularly stressful or otherwise body odor inducing event, such as a meeting at work or a sporting event. The user, potentially not having showered recently (and thus presumably having at least somewhat uncleaned skin in the axilla region) may apply the above-described deodorant formulations to achieve and immediate deodorizing and odor blocking effect. Thus, in general, the methods of use of the presently described deodorant products includes at least the application thereof to either cleaned or uncleaned skill, for example in the axilla region.

Accordingly, the present disclosure has provided deodorant products that effectively counteract the unwanted odors caused by bacteria in perspiration. Additionally, the present disclosure has provided deodorant products that are effective for application and use on skin that has not been recently cleaned, for example between showers. The described deodorant products have been provided in an easy-to-apply form, such as aerosols, pumps, sprays, liquids, roll-ons, lotions, creams, sticks, and soft solids, etc.

While at least one exemplary embodiment has been presented in the foregoing detailed description of the disclosure, it should be appreciated that a vast number of variations exist. It should also be appreciated that the exemplary embodiment or exemplary embodiments are only examples, and are not intended to limit the scope, applicability, or configuration of the disclosure in any way. Rather, the foregoing detailed description will provide those skilled in the art with a convenient road map for implementing an exemplary embodiment of the disclosure, it being understood that various changes may be made in the function and arrangement of elements described in an exemplary embodiment without departing from the scope of the disclosure as set forth in the appended claims and their legal equivalents.

What is claimed is:

1. A deodorant product comprising:
an odor blocking compound that reduces the perceived intensity of malodors or blocks malodors; and
an antibacterial compound effective to kill bacteria upon application thereto.

2. The deodorant product of claim 1, wherein the deodorant product is provided in stick form and further comprises an anhydrous, hydrophobic vehicle, which includes a volatile silicone and/or high melting point component.

3. The deodorant product of claim 2, wherein the high melting point component is selected from the group consisting of: beeswax, spermaceti, carnauba, bayberry, candelilla, montan, ozokerite, ceresin, paraffin waxes, semi-microcrystalline and microcrystalline waxes, hydrogenated jojoba oil, and hydrogenated castor oil (castor wax).

4. The deodorant product of claim 1, further comprising a fragrance.

5. The deodorant product of claim 1, further comprising an antiperspirant active.

6. The deodorant product of claim 5, wherein the antiperspirant active is selected from the group consisting of: salts of aluminum, zirconium, zinc, and mixtures thereof.

7. The deodorant product of claim 1, wherein the deodorant product is provide in an aerosol spray form and comprises a gaseous propellant.

8. The deodorant product of claim 1, wherein the odor blocking compound is selected from the group consisting of: agrumex, C14 aldehyde, CEDRAMBER®, CLARITONE®, eugenol, Δ-damascone, dihydromyrcenol, dynascone 10, GLOBALIDE®, cis-3-hexenol, hexylcinnamic aldehyde, isoananat, menthyl acetate rf, oryclone special, phenylethyl alcohol, rosaphen, sandolene, tetrahydro linalool, vertocitral, and combinations thereof.

9. The deodorant product of claim 1, wherein the antibacterial compound is selected from the group consisting of: phenolic antibacterial compounds, quaternary ammonium antibacterial compounds, anilides, bisguanidines, and combinations thereof.

10. The deodorant product of claim 1, wherein the odor blocking compound in present in the deodorant product in an amount of about 0.1% to about 10% by weight of the deodorant product.

11. The deodorant product of claim 1, wherein the antibacterial compound is present in the deodorant product in an amount of about 0.1% to about 5% by weight of the deodorant product.

12. A method of deodorizing a malodor comprising the step of:
applying a deodorant product to the skin, the deodorant product comprising an odor blocking compound that reduces the perceived intensity of malodors or blocks malodors and an antibacterial compound effective to kill bacteria upon application thereto.

13. The method of claim 12, wherein the step of applying comprises applying the deodorant product to an axilla region.

14. The method of claim 12, wherein the step of applying comprises applying the deodorant product to cleaned skin.

15. The method of claim 12, wherein the step of applying comprises applying the deodorant product to uncleaned skin.

16. The method of claim 15, wherein the step of applying comprises applying the deodorant product in between showers.

17. The method of claim 12, wherein the deodorant product is formulated as a stick, and the step of applying comprises contacting the stick with the skin.

18. The method of claim 12, wherein the deodorant product is formulated as an aerosol spray, and the step of applying comprises depressing a plunger on a spray container to cause a gaseous propellant to emit the deodorant product from the container in spray form to contact with the skin.

19. The method of claim 12, wherein the odor blocking compound is selected from the group consisting of: agrumex, C14 aldehyde, CEDRAMBER®, CLARITONE®, eugenol, Δ-damascone, dihydromyrcenol, dynascone 10, GLOBALIDE®, cis-3-hexenol, hexylcinnamic aldehyde, isoananat, menthyl acetate rf, oryclone special, phenylethyl alcohol, rosaphen, sandolene, tetrahydro linalool, vertocitral, and combinations thereof.

20. The method of claim 12, wherein the antibacterial compound is selected from the group consisting of: phenolic antibacterial compounds, quaternary ammonium antibacterial compounds, anilides, bisguanidines, and combinations thereof.
